(19) United States
(12) Patent Application Publication
(10) Pub. No.: US 2023/0165459 A1
(43) Pub. Date: Jun. 1, 2023

(54) APPARATUS AND METHOD FOR DIMENSIONAL MEASURING AND PERSONALIZING LENS SELECTION

(71) Applicant: EYELATION, INC., Tinley Park, IL (US)

(72) Inventor: Bradley Kirschner, Tinley Park, IL (US)

(21) Appl. No.: 18/058,540

(22) Filed: Nov. 23, 2022

Related U.S. Application Data
(60) Provisional application No. 63/282,405, filed on Nov. 23, 2021.

(51) Int. Cl. A61B 3/11; A61B 3/12; A61B 3/14; G06T 7/62

(52) U.S. Cl. CPC A61B 3/111; A61B 3/14; A61B 3/1216; G06T 7/62; G06T 2207/30041

(57) ABSTRACT

A device for measuring pupillary distance can include a processor for processing an electronic image, such as a selfie. The processor can be configured to determine an iris width of one or both of the eyes. The pupillary distance between the eyes can be determined based, at least in part, on the iris width.

### PRIORITY

This application claims priority to U.S. Provisional Pat. Application No. 63/282,405, filed Nov. 23, 2021, which is incorporated by reference in its entirety.

### TECHNICAL FIELD

The present disclosure relates to measurement devices and, in particular, to a system that measures and facilitates the determination of the pupillary distance. The disclosure also relates to systems and methods that can interface with users’ devices to aid individuals in selecting the proper and/or desired lenses and/or frames.

### BACKGROUND

The potential for unwanted prismatic effect (e.g. induced prism) is often considered when making and/or fitting prescription eyeglasses. Typically, when a subject presents a prescription to an optician, the optician must ensure that the optical center (i.e. the center of vision in the lens) aligns with the subject’s eye along the visual axis of the eye. If misaligned, unwanted prismatic effects can cause asthenopia (eyestrain or ocular fatigue), headaches, etc. Induced prism becomes stronger in proportion to prescription strength, which is shown by Prentice’s rule:

$$P = \frac{c ∗ f}{10}$$

Where, P is the power of the prism in prism diopters, f is the lens power in diopters, and c is the decentration in millimeters, i.e. the distance between the lens’s optical center and the pupil’s center).

A key metric for ensuring proper alignment, as well as other aspects of making and/or fitting glasses and frames, is pupillary distance (PD). This value is also sometimes referred to as interpupillary distance (IPD) or anatomical interpupillary distance, and is generally considered as the distance between the centers of the entrance pupils of the eyes. For various reasons, pupillary distance is often not included in a patient’s prescription.

Differing techniques have been employed for measuring pupillary distance. For example, an optometrist might use a millimeter ruler to measure the distance between the centers of the subject’s pupils. This, however, is prone to several drawbacks, ranging from simple human error to not accounting for parallax, to shortcomings of the tools. Technological advances have been employed to measure PD from an image of a subject. Such systems have been lacking, however. For example, some have relied on using a reference (such as a fiducial or standard sized credit card) in the image to measure relative PD and then calibrate the relative measurement based on the known reference’s measurements. Some have relied on knowing the precise distance of the plane of the subject’s eyes to the camera, and then using that known distance to extrapolate the PD from the relative PD in the image. None of the prior techniques have been able to measure actual pupillary distance from an image of a subject without having some additional known measurement (such as a fiducial).

### SUMMARY

An aspect can include a device for measuring a pupillary distance. The device can include a processor for processing an image. The image can be an electronic image. The image can include an OS eye and/or an OD eye. The processor can be configured to determine an iris width of the OS eye and/or the OD eye. The processor can be configured to determine the pupillary distance between the OS eye and the OD eye based, at least in part, on the iris width.

In some embodiments, the processor can be configured to determine the pupillary distance based on a bowtie measurement. A bowtie measurement can include ten or more iterations. For example, there can be 20 iterations. There can be an arbitrarily high number of iterations. In some preferred embodiments, the number of iterations can be between 20 and 500 iterations. In other preferred embodiments, the number of iterations can be between 20 and 100 iterations. In yet other exemplary preferred embodiments, the number can be between 30 and 50 iterations.

In some embodiments, the processor can be configured to remove outlier data. Outlier data can be removed through, for example, data smoothing or averaging. In some embodiments, outlier data can be removed through application of an elliptical blacking-out of a portion of an image.

In other embodiments, a pupillary distance can be determined based on the formula, such as, d = aR - bR², where d is the iris width, a and b are coefficients, and R is the ratio of the pupillary distance to the iris width. In a preferred embodiment, coefficient a can bes between 12.7 and 13.9, and b can be between 0.08 and 0.12.

Another aspect can include a method for measuring pupillary distance. The method can include receiving an electronic image, aligning the image, iteratively measuring iris widths, determining pupillary distance, and returning the pupillary distance in real-world units, such as millimeters. The electronic image can include an OS eye and an OD eye. Each eye can, of course, include a pupil. The pupils of the OS eye and the OD eye can be aligned along a row of pixels in the image. Diagonal pixel widths of one of the pupils can be iteratively measured. Such measurements can be taken as n rows below and n rows above the row of pixels on which the pupils are aligned. The pixel distance n from the alignment row can change after each iterative measurement. For example, n can be +1 for each iteration, meaning each pixel row is utilized, up to a maximum n. Or, n can be +2 for each iteration, meaning every other pixel row is utilized. The pupillary distance can be determined based on the iteratively measured diagonal pixel widths.

In some embodiments, the pixel width can be determined based on a change in brightness. For example, when traveling pixel-by-pixel from the edge of the image to the center of the image, a drop in brightness from light to dark can indicate the iris’ edge, whereas when traveling from the center of the image toward the edge of the image, and increase in brightness from dark to light can indicate the opposite edge of the iris.

In other embodiments, the iteratively measured diagonal pixel widths can be averaged. Averaging can be utilized to, for example, remove outlier data.

In yet other embodiments, a black-out ellipse can cover at least a portion a pupil. Application of an ellipse can be utilized to remove outlier data and improve processing results, for example by addressing lens flare, unwanted reflections, and other aberrations.

Yet another aspect can include a system for measuring a pupillary distance. The system can include a processor, an API, and physical memory. The processor can be communicatively coupled to a server. The API can be configured to receive an electronic image. In some embodiments, the image can be a selfie-style photograph. The physical memory can be communicatively coupled to the processor. The memory can include software. The software and the processor can be configured to measure the pupillary distance based on an iris width. The measurement of pupillary distance can be taken directly from an electronic image.

In an embodiment, a processor can be configured to determine the pupillary distance based on a bowtie measurement. A bowtie measurement can include various numbers of iterations, as further discussed and described herein. Examples can include 1-2000 iterations, but preferred embodiments can be in the range of 20 to 200 iterations. In other embodiments, a bowtie measurement can determine pixel widths of an iris based on changes in brightness. In some embodiments, the processor can be configured to smooth the iris width and to remove outlier data.

In some embodiments, a system can include a database of prescription data. An API is configured to receive a prescription and analyze the prescription through logic puzzles to build a full prescription.

Yet other embodiments can include a database of prescription data. A system can be configured to receive a prescription and/or return a recommendation of available eyewear frames based on the pupillary distance and the prescription. A pupillary distance (PD) can be determined based on a ratio of iris width to PD. For example, such measurement can be based on a formula such as, d = aR - bR², discussed and described herein.

### BRIEF DESCRIPTION OF THE DRAWINGS

The present invention is further described in the detailed description, which follows, in reference to the noted plurality of drawings by way of non-limiting examples of certain embodiments of the present invention, in which like numerals represent like elements throughout the several views of the drawings, and wherein:

FIG. 1 represents a selfie photograph.

FIG. 2 illustrates a system embodiment and external user devices.

FIG. 3 represents a user-submitted selfie photograph.

FIG. 4 represents a photograph corrected for tilt.

FIG. 5 illustrates a cropped photograph.

FIG. 6 illustrates an eye.

FIG. 7 illustrates an eye cropped and converted for analysis.

FIG. 8 illustrates analysis of an eye.

FIG. 9 illustrates analysis of an eye.

FIG. 10 illustrates analysis of an eye.

FIG. 11 illustrates measurement of pupillary distance.

FIG. 12 depicts graph of empirical results and analysis of iris measurements.

FIG. 13 illustrates a pair of glasses and their optical centers.

FIG. 14 illustrates decentering necessary for a particular wearer of eye glasses.

FIG. 15 represents a flow for deciphering prescriptions.

### DETAILED DESCRIPTION

A detailed explanation of the system, method, and exemplary embodiments of the present invention are described below. Exemplary embodiments described, shown, and/or disclosed herein are not intended to limit any claim, but rather, are intended to instruct one of ordinary skill in the art as to various aspects of the invention. Other embodiments can be practiced and/or implemented without departing from the scope and spirit of the invention.

A system according to embodiments of the present invention can include a set of tools that provide a set of microservices. The microservices can include measuring pupillary distance of a subject based on the subject’s photograph. The microservices can include parsing a prescription and generating viable and/or suggested prescription glasses. The microservices can be utilized by various means, including through an application programming interface (API). The tools can be at a variety of endpoints accessible by a URL, such as tools.eyelation.com. While the system can be the set of tools, the system can also include a broader set of applications, and the microservice can be implemented as a standalone feature.

Highly accurate pupillary distance (PD) measurements can be obtained. But prior systems and techniques all have inherent shortcomings. For example, some systems rely on precisely positioning the camera and the subject at a known distance. Any deviation from the expected distance leads to mismeasurement. Systems that require external references of known size require (1) that the subject have the actual reference at the time of measurement and (2) that the reference and the subject are positioned accurately with respect to one another. Systems have been developed that utilize two cameras that can measure PD based on parallax and trigonometric calculation, can yield highly accurate and precise measurements. But, such systems have only been implements to date with purpose-built cameras.

Based on the fulfilment of tens of thousands of prescriptions, it has been discovered that PD measurements of a single adult subject by two different doctors sometimes leads to two separate PD measurements, meaning that at least one of the measurements is likely incorrect. Such occurrences are not uncommon, and the differences between measurements can be appreciable. Moreover, there are instances where a single subject has different measurements from the same doctor. It has also been discovered that there is a spectrum of precision among doctors’ PD measurements.

There is a somewhat narrow range of iris widths of adults. This measurement is known in the optical industry as horizontal visible iris diameter (HVID). The range is roughly about 11.5 to 12.5 mm. It has been discovered that, regardless of age, gender, ethnicity, etc., the vast majority of people iris width follows closely with the ratio of pupillary distance to iris width. As further discussed herein, iris width can be utilized to calibrate images for PD measurements without the need for external references or specialized cameras. And it has been discovered that image calibration based in part on the small range of the ratio of PD to iris width can be effective in over 92% of subjects for PD measurement and decentering of eyeglasses.

An aspect of present embodiments significantly improves the state of the art. For example, unlike previous attempts, present embodiments can accurately and precisely measure biometric data from what is commonly referred to as a “selfie,” i.e. a self-portrait from a handheld device, such as a smartphone or a point and shoot camera. The need for external references, such as fiducial marks or credit cards, has been eliminated, as has reliance on parallax from two camera angles. Further, precise positioning of the camera and the subject is not necessary. Moreover, the PD measurement is repeatable.

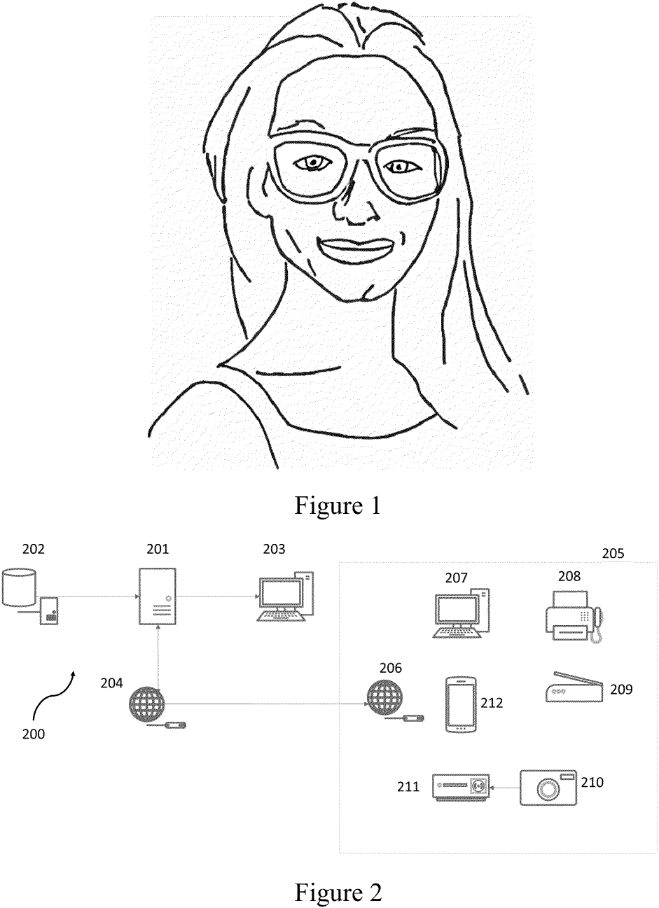

FIG. 1 represents a typical selfie image. The subject is primarily square to the camera, and the face is somewhat tilted. Under ideal circumstances, the subject would be exactly square to the camera and the eyes would be horizontal. Nevertheless, some variation can be accounted for by present embodiments. As discussed further herein, the subject has a “near” PD because the photograph was taken by the subject within her arm’s length at most. Measurements based on selfie images can account for the near PD of the subject.

FIG. 2 illustrates an implementation for easily and accurately obtaining PD measurements and/or prescription glasses. The system (200) can include a server (201) and a database (202). The system can include general purpose computers (203). The system can be connected to a larger network, such as the internet (204), for communication with user devices (205). Generally, user devices can be of any form capable of communicating data over a computer network (206), such as a personal computer (207), a fax machine (208), a scanner (209), a camera (210) and network-connected external drive (211), or smartphone (212). The system can be implemented with an API for advanced users and/or application-based for general consumers.

For example, the user devices (205) can include an app that can execute operations on the user device, such as capturing, saving, and/or sending images. The user devices can be configured to support Hypertext Transfer Protocol (HTTP) for transmitting hypermedia documents, such as HTML. Such operations can run inside the app’s memory space or in an external module that can be driven by the app. Other operations, such as determining PD, can run on the system (200) side. The specific operations running client-side versus system-side can be optimized based on an architect’s preferences such as practical considerations of typical device memory and power, data security, computational needs, size and control limitations for apps, user experience requirements, etc.

An advantage of the system and method embodiments is that the user is not tied to any particular hardware. For example, anyone with an iPhone or Android phone can take a selfie and upload the picture to the PD measurement system with their prescription. Further, those in the industry can access the PD measurement system through an API from whatever computer environment they are using.

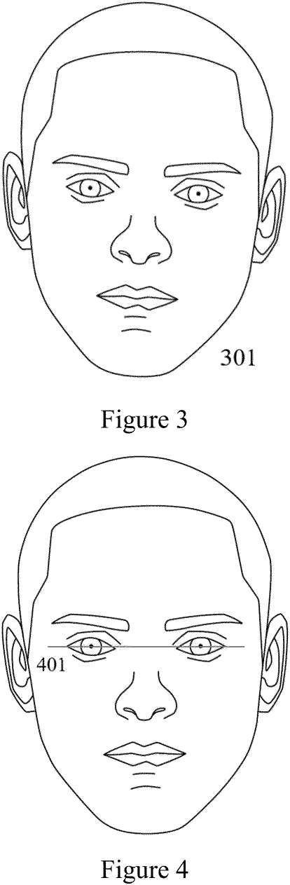

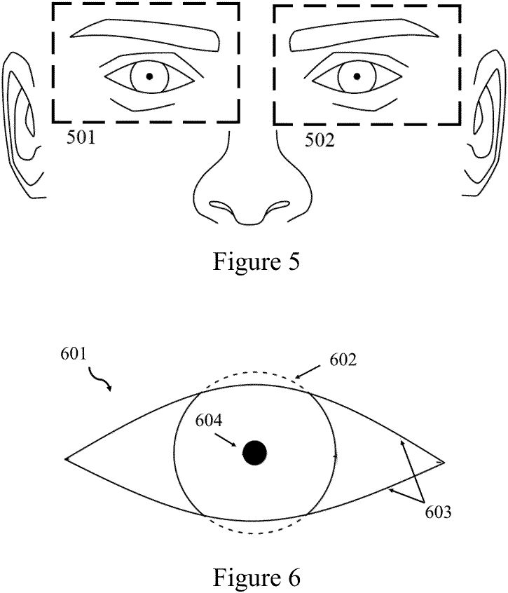

FIGS. 3 through 14 generally relate to measuring PD according to some embodiments. FIG. 3 illustrates a user-provided image (301) that is received by the system (200). As shown, the selfie is slightly askew. The system can rotate the image so that it is square. For example, as represented in FIG. 4, the system can create a copy of the original image and rotate the image in the new file so that the pupils of the subject are aligned with a single row of pixels (401). After the face is rotated, the system can create cropped images (501, 502) of each of the aligned eyes, as represented in FIG. 5. Each of these steps is discussed in greater detail through descriptions of various embodiments hereinbelow.

FIG. 6 illustrates a cropped image of the subject’s eye (601). As is common, the subject’s iris (602) is partially concealed by the eyelids (603). As discussed in greater detail herein, a partially covered iris can raise challenges in measuring PD, but present embodiments have overcome such challenges. In particular, a “bowtie” measurement method can be utilized. Further, eyes are not perfect circles. Publicly available tools, such as the Hough gradient method have been thoroughly tested but have shown to be very inconsistent when measuring iris width. The bowtie process described herein can compensate for a lack of uniformity in geometry. The system implementing the bowtie process for measuring PD can center the subject’s pupil (604) in the cropped image.

After cropping, the two eye images can be converted to grayscale and the images can be adjusted to have high contrast to improve the definition of edges in the images.

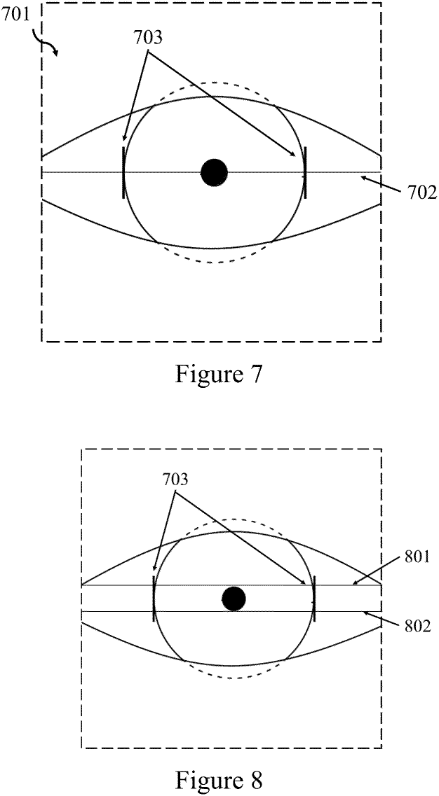

As shown in FIG. 7, the system can identify a row of pixels (702) passing through the pupil in the cropped image. The system can center a square (701) on the pupil in each cropped image. Starting with the central row of pixels (702), the system can identify edges of the iris (703) in the black and white images. For example, the system can sample each pixel from center to edge of the square to create a histogram. The system can identify the largest change in dark to light in the histogram. The system can repeat the sampling from the center to the opposite edge of the square and similarly identify the largest change in dark to light. The measurements can populate an iris edge array.

Highlights and/or reflections on an iris in an image can skew captured data. Processing images with various lighting conditions can nevertheless be achieved. For example, when the system looks for areas or largest histogram change, a blacked-out ellipse can be applied starting from the center of where an eye is detected. The blacked-out overlay can prevent highlights and reflections from interfering with the iris edge detection process. A blackout process can also improve analysis of very light-colored irises (blue, green, etc.). Various radii can be utilized to generate the ellipse overlay. For example, the major axis can be vertical or horizontal, the axes can be rotated, and/or the major and minor axes can be equivalent. A radius of approximately 40 pixels has been shown empirically optimal, providing the most consistent results, for processed images having the 1000-pixel image resize check (described herein) applied. In a preferred embodiment, the blackout process is applied only if the subject distance is determined to be near the camera (i.e. only for near PD).

As shown in FIG. 8, the system can move up one pixel row (801) and down one pixel row (802) and repeat the process of measuring dark to light to identify the iris edge in each horizontal direction. The pixel rows shown in FIG. 8 are representative and obviously not to scale. The measured iris edge can further populate the iris edge array.

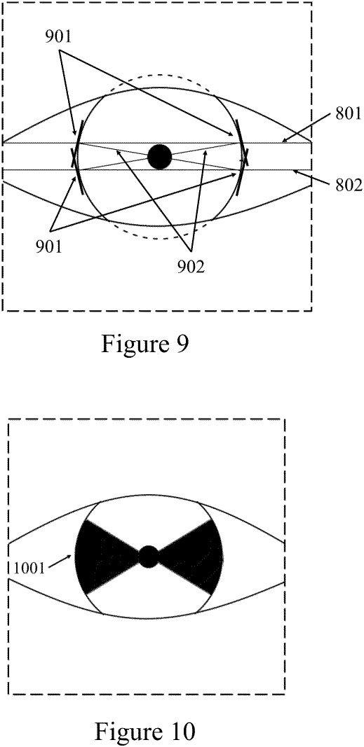

FIG. 9 shows the four detected iris edges (901) at the upper and lower pixel lines (801, 802). Based on the measured iris edges, the system can measure the iris diameter (902) in pixel units from the left lower side to the right upper side and from the left upper side to the right lower side.

As shown in FIG. 10, as the number of iterations increase, the overlapping diameter lines create what looks somewhat like a bowtie (1001), hence the name “bowtie” process. Accurate iris measurements have been obtained with as few as 20 iterations. The process is fast, and the number of iterations can be arbitrarily high, substantially limited only by the number of pixel rows passing through the iris. The number of rows sampled can be automated, for example through optimization, or predefined. Further, not every row of pixels need be sampled. For example, every other row can be sampled. Other numbers of rows can be skipped, according to user preference and needs. For example, every third, tenth, fiftieth, etc. rows can be sampled. Here too, the number of rows to skip can be predefined or automated.

It can be advantageous to have a robust set of iris measurements. For example, differences in the edge of the iris can be smooth (for example, through averaging). Outlier measurements can be discarded. Outliers can occur from different image defects, such as lens flare and reflections in the eyes, both of which can be sampled around. For example, if a threshold percentage of measurements are inconsistent with the majority of measurements, the system can recognize a defect in the image and compensate by omitting outliers from the final iris measurement.

One cause of outliers is the shadow cast from the eyelid in certain lighting conditions that result in lengths far past the edge of the iris. Such outlier data, and other outliers, can be appropriately removed by, for example, relying on known human ranges. As an example, greater than 99 percent of PD ranges fall on or between approximately 54 mm and 75 mm. Thus, outlier data can be excluded for measurements that, if applied in an iris ratio to calculate a PD, fall outside the range. Optimization can include mode analysis to handle outlier data. For example, a data scrub process can determine the mode of values returned and remove extreme outlier data to improve consistency for averages.

Identification of image defects can be useful. For example, they can be used to optimize the number of pixel rows sampled. In some embodiments, the system can begin with sampling the center row. The detected iris edge can be used to create an upper bound for pixel rows to sample above and below the center line. The upper bound can be less than the pixel length of the first-row measurement given the likelihood of the top and bottom of the iris to be concealed in the image, The system can sample ten (for example) evenly-spaced pixel rows above and below the center row of pixels, accounting for the upper bound. If no defects are discovered and/or if the iris measurements are sufficiently close to an expected arc, then subsequent iterations need not be taken. In other words, if there are sufficiently low occurrences of outliers and/or the measurements are all within an acceptable threshold for variance or dispersion, the system can have a level of confidence that further measurements are unlikely to significantly improve the iris measurement. This example of 21 sampled rows is only an example. Because of the relatively little computing power needed to detect iris edge in an image, the actual number of sampled rows can be significantly higher with minimal expected impact on the speed of the overall measurement. For example, in a recent analysis of 67 subjects, the average iris width was 255.4 pixels. The time and computational burden can be trivial for sampling about 260 pixel rows and calculating iris width.

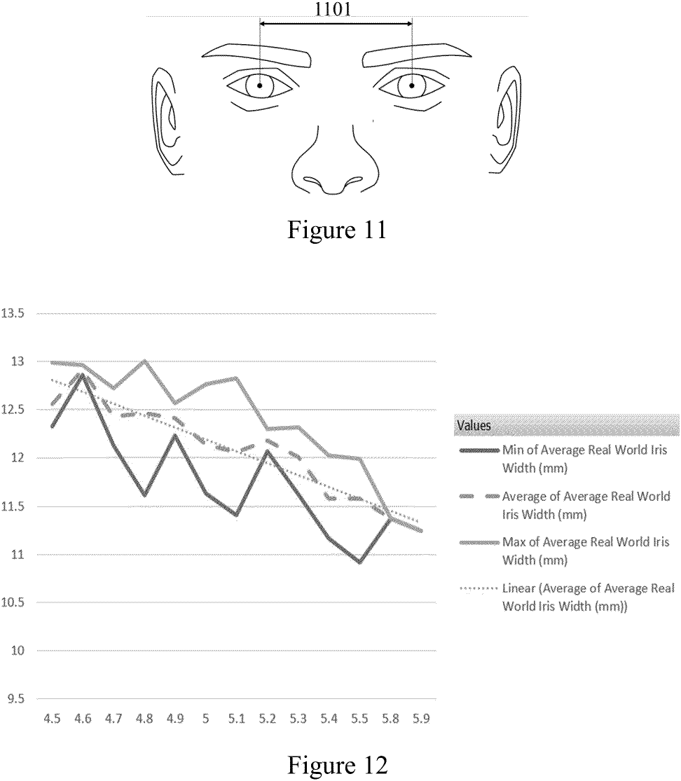

As illustrated in FIG. 11, the system can measure the pupillary distance in pixels (1101). For the reasons discussed above, this information on its own is not tethered to real-world units of distance. However, it can be calibrated based on the pixel measurement of iris width in the image in combination with an empirical ratio described herein.

FIG. 12 shows empirical plots and a trend line. The abscissa is Ratio, the ratio of PD to iris diameter for a pool of subjects. The ordinate is y, the real-world iris diameter in millimeters for the subjects. The trend line is represented by the empirical formula:

$$y = \left(\left(- 0.1 \times R a t i o\right) + 12.8\right) \times R a t i o$$

The plots in the figure are based on 67 subjects chosen to provide a relatively diverse pool. It is noted, however, that based on further analysis the formula remains sufficiently accurate for the desired purpose in more than 92% of cases. Increasing the pool size does not significantly impact the formula, either adversely or positively. Outliers are typically the result of eyes being farther apart than normal in conjunction with irises being larger or smaller than normal. In such cases, secondary data can be utilized to improve results. Based on basic algebra, the same formula can be recast, for example, as a quadratic with coefficients:

$$y = a \times R a t i o - b \times R a t i {o}^{2}$$

It is also noted that while the constants in the formula above are -0.1 and 12.8, other constants can be used. For example, any number in the range of about 11 to 14 can be suitable for the coefficient a. For the avoidance of doubt, both expressions of the formulae above are equivalent and are obviously mathematical abstractions of how a computational system actually computes data, as one of ordinary skill would appreciate.

With the real-world iris width determined, the PD in pixel units can be converted to real-world PD in millimeters. With the real-world PD, the proper amount of decentering can be determined.

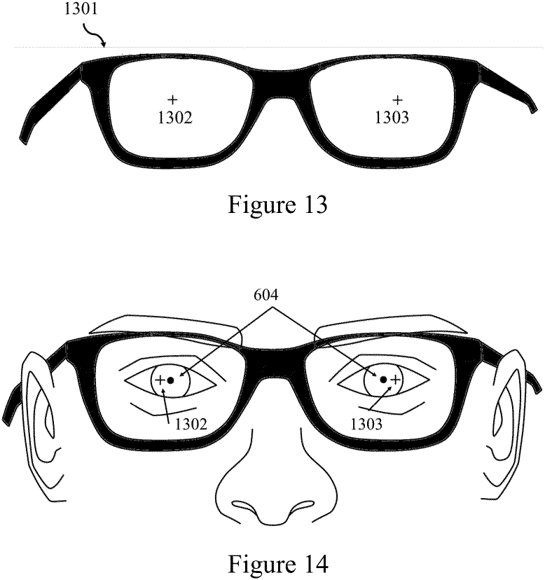

FIG. 13 illustrates a pair of prescription glasses (1301). The right (OD) lens has its optical center (1302) as indicated by a cross. Similarly, the optical center (1302) of the left (OS) lens is indicated by a cross.

FIG. 14 illustrates the glasses of FIG. 13 when worn by the subject. The subject’s pupils (604) are not aligned with the optical centers of the glasses. As discussed above, this results in unwanted prismatic effect. The PD measurement system can calculate the amount of decentering required for the subject and a given set of eyewear. This can be advantageous for reasons beyond just decentering. For example, the system’s database can maintain specifications for frames and available lens blanks. Based on those specifications, the system can determine whether the amount of decentering is within acceptable parameters. The system can further make recommendations and/or provide a list of suitable frames based on the amount of decentering. For example, if the subject desires a particularly small or rather large glasses, but the amount of decentering would be outside acceptable parameters, the system can caution the user against the choice (or simply not provide such choices).

It should be understood that, in the context of eyeglasses, “frames” is often used in the singular sense—e.g., frames for a pair of glasses-and in the plural sense—e.g., several frames. The broadest sense of the term is contemplated herein, unless otherwise stated or where context makes clear a particular instance of the term is meant as specifically singular or plural.

Embodiments can meet or exceed allowed tolerance for induced prism under ANSI 780-1 (2015) - The Vision Counsel Quick Reference Guide. For example, the prism reference point is not allowed to be more than 1.0 mm away from its specified position in any direction. Measuring to precision of 1.0 mm with a physical ruler can be difficult to achieve. While such precision is not really necessary for weaker prescriptions, such as -1 Diopter, that level of precision certainly becomes necessary at larger prescriptions. Because embodiments described herein can achieve better than the ANSI standard, they can be of greater advantage for stronger prescription strengths.

Parameters and assumptions can be utilized for improving PD measurements. For example, as mentioned above, there are two types of PD, far and near. Far PD refers to the distance between the pupils when a person is viewing a distant object. Eye doctors typically use this type of PD for distance vision glasses. Near PD is the distance between the pupils when an individual is looking at a near object, such as when reading. The standard reading distance is around 16 inches. This is also about the same distance at which individuals typically take selfies for uploading to the PD measurement system. In such cases a normal correction of 3.5 mm can be acceptable. For larger the PDs, the near PD measurement can be adjusted more. If the camera is far from the subject, a correction of 1.5 mm can be acceptable. The relative face size in the digital image can be used to categorize the subject’s distance. For example, categories of very near, near, near to intermediate, and far can be sufficiently accurate. Corrections of 1.5 to 4.5 millimeters can be sufficiently accurate. Experience also shows that cropped photographs or otherwise altered can lead to improper correction for near and far PD.

Subject distance PD adjustments can be made. Standard adjustments used in the optical industry are shown in Table I. But further refinements can be made. In a preferred embodiment, for example, refined the adjustments are also shown in Table I.

TABLE I

| | | |
|---|---|---|
| Subject Distance | PD Adjustment (Standard) | PD Adjustment (Refined) |
| Very Near | PD ≤ 57 add 2.5 57 < PD ≤ 67 add 2.5 PD > 67 add 4.5 | No adjustments |
| Near | No adjustments | No adjustments |
| Intermediate | PD ≤ 57 add 1.5 57 < PD ≤ 67 add 2.5 PD > 67 add 3.5 | PD ≤ 57.5 add 1.5 57.5 < PD ≤ 68.5 add 2.5 PD > 68.5 add 3.5 |
| Far | No adjustments | No adjustments |

Techniques, methods, and systems described herein can be implemented in part or in whole using computer-based systems and methods. For example, a computer can implement the algorithms and perform functions directed by programs stored in a computer-readable medium. Embodiments can take the form of hardware, software, or a combination of software and hardware. Embodiments can take the form of a computer-program product that includes computer-useable instructions embodied on one or more computer-readable media. Additionally, computer-based systems and methods can be used to augment or enhance the functionality, increase the speed at which the functions can be performed, and provide additional features and aspects as a part of or in addition to those described herein.

Such hardware can include a general-purpose computer, a server, network, and/or cloud infrastructure and can have internal and/or external memory for storing data and programs such as an operating system (e.g., DOS, Windows 2000™, Windows XP™, Windows NT™, Windows 7™, Windows 8™, Windows 8.1™, Windows 10™, OS/2, UNIX, Linux, Android, or iOS) and one or more application programs. Examples of application programs can include computer programs implementing the techniques described herein for customization, authoring applications (e.g., word processing programs, database programs, spreadsheet programs, or graphics programs) capable of generating documents or other electronic content; client applications (e.g., an Internet Service Provider (ISP) client, an e-mail client, short message service (SMS) client, or an instant messaging (IM) client) capable of communicating with devices, accessing various computer resources, and viewing, creating, or otherwise manipulating electronic content; and browser applications (e.g., Microsoft’s Internet Explorer) capable of rendering standard Internet content and other content formatted according to standard protocols such as the HTTP. One or more of the application programs can be installed on the internal or external storage of the general-purpose computer. Alternatively, application programs can be externally stored in or performed by one or more device(s) external to the general-purpose computer.

The computer preferably includes input/output interfaces that enables wired and/or wireless connection to various devices. In one implementation, a processor-based system of the general-purpose computer can include a main memory, preferably random access memory (RAM), and can also include secondary memory, which may be a tangible computer-readable medium. The tangible computer-readable medium memory can include, for example, a hard disk drive or a removable storage drive, a flash-based storage system or solid-state drive, a floppy disk drive, a magnetic tape drive, an optical disk drive (Blu-Ray, DVD, CD drive), magnetic tape, standalone RAM disks, drive, etc. The removable storage drive can read from or write to a removable storage medium. A removable storage medium can include a disk, magnetic tape, optical disk (Blu-Ray disc, DVD, CD) a memory card (CompactFlash card, Secure Digital card, Memory Stick), etc., which can be removed from the storage drive used to perform read and write operations. As will be appreciated, the removable storage medium can include computer software or data.

In alternative embodiments, the tangible computer-readable medium memory can include other similar means for allowing computer programs or other instructions to be loaded into a computer system. Such means can include, for example, a removable storage unit and an interface. Examples of such can include a program cartridge and cartridge interface (such as the found in video game devices), a removable memory chip (such as an EPROM or flash memory) and associated socket, and other removable storage units and interfaces, which allow software and data to be transferred from the removable storage unit to the computer system.

The server can include the general-purpose computer discussed above or a series of containerized applications running on commodity or cloud-hosted hardware. The SDF can be implemented within a network, for example, the Internet, the World Wide Web, WANs, LANs, analog or digital wired and wireless networks (e.g., Public Switched Telephone Network (PSTN), Integrated Services Digital Network (ISDN), and Digital Subscriber Line (xDSL)), radio, television, cable, or satellite systems, and other delivery mechanisms for carrying data. A communications link can include communication pathways that enable communications through one or more networks.

Returning to FIG. 2, while the system (200) can be implemented as shown, it can alternatively be implement on a single general-purpose computer or any computing environment. Some embodiments can implement an automated prescription verification module, Check Rx, and/or a virtual try on module. In a further example of an embodiment alternative, the PD measurement system and/or Check Rx can be implemented entirely in a downloadable app. The app can be downloaded to user devices (205)—such as smart phones, laptops, note pads, etc.-and/or other devices-such as point of sale terminals, kiosks, etc.

When a user accesses the system (200), the system can first check whether the system has current prescription data for the subject. If not, the system can invoke the Check Rx module, which can process a user’s new prescription. The system interface can provide for user inputs. If a user inputs the prescription information, Check Rx can check for detectable errors, which are discussed further below. Check Rx can also be implemented with a digital reading tool. This can allow a user to submit a scan or image of the doctor’s prescription. This can be particularly convenient for the user. For example, the user can take a selfie and take a picture of the prescription and send them both to the system for analysis.

The user can have an account with the supplier of the system and/or app. In a web-based and in an app-based implementation, the user can create login credentials. After creating an account and logging in, the user can edit personal details. For example, the user can indicate information such as age, birthdate, gender, whether the user has previously worn glasses, whether the user will wear the glasses fulltime or only for specific activities, whether the glasses also need to serve as safety glasses, etc. The information can be used to make recommendations, such as frame types, lens coatings, and/or whether or not to tint a lens.

In an API-based implementation, the professional user can obtain a key and access the system via the user’s preferred means of communicating through the API endpoints, which can be URLs. In a preferred embodiment, the system can include two endpoints for accessing the API, CheckRx and ReportRx. In some embodiments, however, the professional user can create an account. After creating an account and logging in, the user can create patient profiles and submit patient data, such as the personal details above, as well as prescription data.

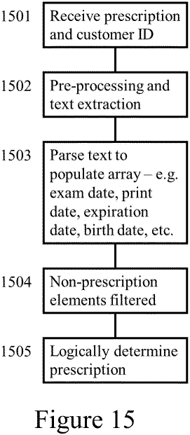

In a preferred API-based embodiment, a user submits an image of a prescription and a customer ID via the CheckRx endpoint, along with the API key. FIG. 15 shows an exemplary embodiment, in which the system can receive (1501) an image of a prescription and a customer ID. Check Rx can preprocess the image and extract text (1502). Text blocks can be returned, and handwritten text can be ignored. Returned text block can be parsed (1503) for dates to populate an array for exam date, print date, expiration date, birth date, etc. The response can be filtered (1504) to remove any elements that are not part of the prescription (such as letters or certain characters in the detected block that can cause removal with some exceptions). Lens axes can be found as numerical values of three characters or less. Other prescription values, such as sphere and cylinder can be determined from prescription items having more than three characters and that are numerical.

After parsing and extraction (1502), the prescription can typically be determined (1505) through logic puzzles, for example based on the number of rows found, number of elements found, and/or the existence of one or two axis values. The resulting logic puzzle analysis can be used to build out the full prescription. An example is provided in Table II.

TABLE II

| Event | Description |
|---|---|
| Cannot identify elements Adds do not match | Inconsistency with elements in the prescription. The add powers do not match. This is unlikely although not impossible. This is more likely caused by an error in element identification. A manual review may be performed. |
| Opposite sign cyl | Cylinder powers must be the same sign positive or negative. This is likely caused by an error in the OCR extraction. A manual review may be performed. |
| Negative add power | Add powers cannot be negative. This is likely caused by an error in element identification. |
| Possible PD conflict | It is possible that the PD was mistaken for an axis power. A manual review may be performed. |
| Not enough data | Unable to differentiate if the Rx is for the right or left eye. A manual review may be performed. |
| Too many elements | Too many elements were extracted. Unable to identify the prescription clearly. Possibly multiple prescriptions on the page. A manual review may be performed. |
| Application error | Unable to read image. Possibly wrong format or not a prescription. |

A false positive can be considered a situation where no prescription extractor warnings are displayed, but the prescription data is not entirely correct. Example can be where a system indicates that the right axis is 100 but should be 10, or where a prescription shows an ADD but no data is extracted. Mismatched or incorrect exam or expiration dates do not need to be flagged.

The Check Rx module can identify various errors. It can be useful to require checking for the following errors: right axis missing, left axis missing, right axis out of range, left axis out of range, opposite signs cyl, 0.25 steps check, add power strength, sphere power limit, cylinder power limit, PD 0.5 steps check, seg 0.5 steps check, seg out of range PAL, if add needs multifocal, and if multifocal needs add. Checking for other errors can be useful, such as: opposite signs sphere, add power unequal, sphere power differential, cylinder power differential, unequal seg heights, PD differential, seg low for frame with PAL, seg high for frame with Bif, and frame size likely too small.

Various techniques can be utilized to extract the text blocks from the prescription. For example, a subroutine can preprocess the image to binarize, remove noise, rescale, deskew, etc. the image. Next, the preprocessed image can be processed using a tool, such as Tesseract, to recognize the text within the prescription. Python wrappers, such as Pytesseract, are available that can both preprocess and perform optical character recognition (OCR). Additionally, there are API-based tools, such as Adobe’s OCR API and Amazon’s Textract and Comprehend, that can be utilized for identifying the text.

Embodiments can be configured to accept various types of images. Raster images for example can be used and can include JPEG (or JPG), GIF, and PNG files. Other file types can be utilized, including PNG, GIF, TIFF, PSD, PDF, EPS, AI, INDD, and RAW. Numerous methods and tools for converting among file types are known, many of which can be freely downloaded. In a preferred embodiment, part of preprocessing invokes a subroutine to convert a received image to JPEG.

Virtual try-on functionality can be implemented using various off-the-shelf augmented reality platforms. For example, Google, Meta/Spark, and GitHub all have tools for implementing augmented reality, such as ARCore (Google) and Spark AR Studio. The functionality can be implemented in the app and/or through an API. In an API-based implementation, the functionality can be deployed within the system. Alternatively, integrating an API to a third-party platform, such those interfaces found in Spark AR Studio, can significantly reduce upfront development time.

In an embodiment, an original unmodified photo can be uploaded to the API endpoint. It should be understood that the uploading the photo-as well as the discussion of this embodiment-can be implemented in an app-based and/or web-based embodiment. Through the API, the user can be notified that the photo should meet certain parameters, such as: the photo should be a selfie; the photo should be in portrait orientation; the entire face must be in the photo; the eyes of the person in the photo should be fully open; the person should be looking directly at the camera with their face straight ahead and not from the side; the image should not be a picture of another image; and there should only be one person in the photo.

The tools service can save the original image in a unique folder for that user and/or subject. The tools service can save the original image with the filename it was given from the API user or generate a unique filename. Thereafter, copies of the original image can be modified. As an example, the image can be made to be no more than 1000 pixels in width, and the modified file can be saved with a filename such as “resized_<filename>.<ext>”. Image quality of the image can impact data returned from some services, and it can also have an effect on the iris measurements. An improvement can include correcting for inconsistent measurements for the same person (not photo). A resolution check can be applied against the image provided to confirm that no dimension is less than, for example, 1000 pixels, and resizing of the image can be performed if needed. By applying this improvement, grayscale cropped images can be also more consistent in size and quality.

Embodiments can include a facial recognition subroutine. The subroutine can be invoked, for example, through a Face API. The facial recognition subroutine can utilize filters to identify a human face in an image and transform the face into numerical expressions. Based on deep learning, for example, the subroutine can generate various data about the person, such as estimated age and gender, in addition to spatial information. The subroutine can be trained based on machine learning and a data set. However, commercially available facial recognition tools can be integrated (or accessed through APIs) to reduce upfront development time and alleviate the need for large training data sets. It is noted that in some third-party facial recognition APIs, the terms “left eye” and “right eye” can be reversed from their ordinary meaning in medicine. For example, Microsoft’s API uses “pupilRight” for the subject’s left eye and uses “pupilLeft” for the subject’s right eye. While solutions can be trivial, it is nevertheless important to ensure OS (subject’s left eye) and OD (subject’s right eye) are appropriately assigned to “left” and “right” eyes identified by the facial recognition tool. Some third-party tools can introduce error that can be improved upon. For example, circle identification can be utilized to re-center an initial pupil identification from facial recognition APIs.

If a face is not identified in the modified image, then both the resized image and the original image can rotated 90 degrees. Facial recognition can be performed on the rotated and resized image. This can be repeated up to three rotations until a face is found. If after four attempts there is no dataset—i.e. no face is recognized—an error message can be returned to the API user that no face was found. Alternatively, the substantially same identification process can be performed using seven 45-degree rotations or three 90-degree rotations, a 45-degree rotation, and three more 90-degree rotations.

In a preferred embodiment, facial recognition is utilized to populate a data structure. The format of the data structure can be left to an architect’s preferences, but JavaScript Object Notation (JSON) can be a convenient solution. The data structure can include various information that can be utilized by different aspects of the embodiments. An example of a preferred JSON data structure is provided below.

“face” : {
“age_est”: 41,
“binocular_distance_PD”: 62.5,
“clockwiseRotation”: 90,
“file”: “ac2a08482dc86e33.jpg”,
“gender_est”: “male”,
“id”: “12345kisjg”,
“iris_mm”: 11.69,
“iris_px”: 114,
“left_iris_px”: 112.02059,
“pd_pixels”: 573,
“pitch”: -15.2,
“pupils”: {
“left_eye”: {
“x”: 1908.8,
“y”: 955.6
},
“right_eye”: {
“x”: 1335.4,
} “y”: 964.5
},
“ratio”: 5.037,
“relativeFaceSize”: 0.23666,
“right_iris_px”: 115.69384,
“roll”: 0.9,
“subjectDistance”: “Very Near”,
“wears_glasses”: “NoGlasses”,
“yaw”: -1.7
}

The data can be utilized for different functionality and purposes. For example, estimated age can be used to generate a warning message if the subject is estimated to be below a certain age, such as sixteen.

Pupil points can be translated to the original pupil points on the original image, for example by using the ratio of their distance to the edges between images. It should be noted that the pupil placement returned from some API can be imprecise, which can cause issues with the final result. But the system can correct that, for example through a subsequent correct step.

The original image can be rotated such that the eyes are aligned on the same horizontal (y) line. The angle of rotation can be calculated as follows: opposite = OS Pupil X Coordinate – OD Pupil X Coordinate adjacent = OD Pupil Y Coordinate - OS Pupil Y Coordinate rotation_angle = atan(opposite / adjacent) ∗ (180 / π) - 1 The original image can be rotated by the calculated rotation_angle. The center of rotation can be the midpoint between the eyes in the original image. The file can be saved with a new filename, such as “rotated_<filename>.<ext>”, to preserve the original image file.

Eyes can be extracted from the original image and each saved as a separate file, one for OD and one for OS. The inner and outer points can be determined. These points can be translated to their counterpart location on the full-size image by, for example, using the ratio of the placement in relation to the edges of the image. These points can represent the sides of the image that can be cropped to form individual eye images for each eye. A square can be formed from the horizontal distance between those points by cropping vertically by the same dimension. Each eye image can be saved, for example with filenames “OD_<filename>.<ext>” and “OS_<filename>.<ext>”.

The eye images can be converted to grayscale. The contrast and brightness can enhanced to create more obvious separation between the iris and the sclera. The images can be saved as, for example, “OD_grayscale_<filename>.<ext>” and “OS_grayscale_<filename>.<ext>”.

The outer edge of each grayscale eye image can be discarded from consideration to reduce noise from the outer edges of the picture. For example, 5% can be discarded. Other amounts, for example from 0% to 25%, can be discarded according to the architect’s preferences and optimization of noise reduction.

The color of a number of pixels can recorded by the system. For example, starting from the pupil center, the color depth of each pixel outward and inward horizontally can recorded in an array. Where the maximum change from darkness to light is found can be considered the maximum change in color. That pixel can be recorded in another array. The of distance the two pixels between the outer and inner limit of the maximum values can be recorded. The process can be repeated, incrementing one pixel above the pupil center and out, and one pixel below the pupil center and in-with the distance between the pixels where the maximum change in color from dark to light is found. As discussed herein, the number of iterations can depend on preference, confidence, optimization, processing power, and other considerations. However, in a preferred embodiment, about twenty distinct lines across the iris in which distance is recorded can be sufficient. Expecting distances to be within about 45-70% of the total image width for the eye image can also be sufficient. In a preferred embodiment, any value outside of that range is discarded. Most often light reflections in the eye image can cause a false positive cutting off the measurement too soon by showing a large change from dark to light. The remaining distances can be averaged to create an output of average iris width in pixels. Because the eye images are cropped from the original size image, the pixel values are relative to that image and not the reduced image.

A secondary subroutine can be invoked to detect the irises from each eye image using the Hough transform. Publicly available tools, such as OpenCV, can be utilized. For reasons discussed herein, Hough is not part of a preferred embodiment for measuring PD. However, the technique can be useful in some embodiments. For example, the technique can be used as a check for the bowtie PD measurement, and it can be used for aspects other than PD measurement. Using the Hough transform, each full color eye image can be passed to a function that separately converts them again to grayscale and runs them through the Hough process.

This differs from the edge detection method discussed above. For example, the Hough process in OpenCV takes a 3×3 grid of nine pixels and ranks them procedurally, as discussed more fully in docs.opencv.org/master/d3/de5/tutorial_js_houghcircles.html. The output of the process is a circle radius, and placement of the circle center which is the pupil center per the Hough process. The Hough pupil centers are then compared to the centers of the pupils from the original eye images to create a differential between them in x and y coordinates. The final pupil placement is determined then by adding the offset placement to the pupil output from facial recognition to create a final pupil placement in the original image. The radius from the Hough process can be multiplied by two to get iris diameter in pixels. That can be compared against the output of the iris diameter from the bowtie measurement as an additional checkpoint. Although the Hough algorithm can be utilized, for example as an error check, preferred embodiments do not rely on Hough. It is simply not sufficiently precise.

The distance between eyes can be measured in pixels using the distance formula from the final pupil placement after adjustments to the output have been applied. Radius generation logic can be applied to the output of the bowtie measurement. The results of the pixel measurements can be converted to millimeters, as discussed above.

A preferred embodiment can include the following steps listed below. Not all of the steps are necessary. And the steps need not be performed in the following order except where required by logic. For example, Steps 102-104 can be done in any order (depending on system configuration) or can be omitted entirely (depending on system configuration, for example, where the system is not app-based, where the image is not a selfie captured from the user’s mobile device, and/or where the prescription is digital and does not need to be extracted from a paper document).

Step 101: The user obtains a prescription for eyeglasses from an ophthalmologist, optometrist, or other health-care provider.

Step 102: The user accesses an app on their personal mobile device, such as a cell phone, preferably logging on with a user identification and password.

Step 103: The user aligns her face with the camera of the mobile device, preferably square to the camera, and takes a selfie photo.

Step 104: The user aligns her prescription with the camera, preferably square to the camera, and takes a photo of the prescription.

Step 105: The user submits the selfie and prescription image to the system.

Step 106: The system processes the selfie, such as correcting for rotation of the user’s face, if necessary, extracting left eye and right eye images, converting the image files to grayscale, and increasing image contrast.

Step 107: The system measures PD of the user.

Step 108: The system analyzes the prescription.

Step 109: Based on the PD measurement and the prescription analysis, and using a database of frames, the system determines a suggested set of frames and an allowed set of frames.

Step 110: The app provides the user one or more sets of frames to choose from.

Step 111: The user selects a frame, or a set of frames, through the app.

Step 112: The app displays a frame on an image of the user’s face through the mobile device, preferably on a real-time augmented reality image of the user.

Step 113: The app provides categories to assist the user in choosing frames, such as frame style, brand, material, color, shape, and price.

Step 114: The user can save selected frames and return to the app later.

Step 115: The app displays the extracted digital prescription and prompts the user to confirm the accuracy of the digital information.

Step 116: The app displays recommendations for frames.

Step 117: The user purchases the prescription eyeglasses and frame through the app.

Step 118: The system provider purchases the selected frame or picks the selected frame from previously-purchased stock, causes the lenses to be manufactured and applied to the selected frame, and causes the assembled eyewear to be shipped to the user.

Embodiments herein are discussed primarily within the contexts of PD measurements and fitting of eyeglasses. Various embodiments, however, have broader applicability. For example, the bowtie process can have broader applicability to machine analysis of images. The Hough transform discussed above uses a procedure of binning votes on pixels to identify an object in an image-particularly in identifying round objects. But it requires a high number of votes to fall in the right bins, else Hough becomes very inefficient and loses accuracy over background noise. Hough does not work well for partial shapes, for example. The bowtie process described herein can compensate for a lack of uniformity in geometry. The bowtie process can be implemented to achieve an efficient way of determining, for example, radius of curvature and roundness of whole and/or partial geometric shapes in images. The bowtie process is also more efficient, using less computer processing power, than the Hough transform. It is also noted that the bowtie process is discussed above using a horizontal orientation, but the process can be performed vertically or in any orientation.

All of the methods and systems disclosed herein can be made and executed without undue experimentation in light of the present disclosure. While the apparatus and methods of this invention have been described in terms of preferred embodiments, it will be apparent to those of skill in the art that variations may be applied to the methods and in the steps or in the sequence of steps of the method described herein without departing from the concept, spirit and scope or the invention. In addition, from the foregoing it will be seen that this invention is one well adapted to attain all the ends and objects set forth above, together with other advantages. It will be understood that certain features and sub-combinations are of utility and may be employed without reference to other features and sub-combinations. All such similar substitutes and modifications apparent to those skilled in the art are deemed to be within the spirit and scope of the invention.

We claim:

1. A device for measuring a pupillary distance, comprising:
a processor for processing an image, wherein the image is an electronic image comprising an OS eye and an OD eye;
the processor being configured to:
determine an iris width of the OS eye or the OD eye, and
determine the pupillary distance between the OS eye and the OD eye based on the iris width.

2. The device of claim 1, wherein the processor is further configured to determine the pupillary distance based on a bowtie measurement.

3. The device of claim 2, wherein the bowtie measurement includes ten or more iterations.

4. The device of claim 1, wherein the processor is further configured to remove outlier data.

5. The device of claim 1, wherein the pupillary distance is determined based on the formula:
$$d = a R - b {R}^{2}$$
where d is the iris width, a and b are coefficients, and R is the ratio of the pupillary distance to the iris width.

6. A method for measuring pupillary distance, comprising:
receiving an electronic image of an OS eye and an OD eye, wherein each eye contains a pupil;
aligning the pupils of the OS eye and the OD eye along a row of pixels;
iteratively measuring diagonal pixel widths of one of the pupils from n rows below to n rows above the row of pixels, where n changes after each iterative measurement;
determining the pupillary distance based on the iteratively measured diagonal pixel widths; and
returning the pupillary distance in real-world units.

7. The method of claim 6, wherein the pixel width is determined based on a change in brightness.

8. The method of claim 6, further comprising averaging the iteratively measured diagonal pixel widths.

9. The method of claim 8, further comprising removing outlier data.

10. The method of claim 6, wherein a black-out ellipse is laid over at least a portion of one of the pupils.

11. The method of claim 6, further comprising:
receiving a prescription and a customer ID;
preprocessing and extracting text from the prescription;
parsing text of the prescription to populate an array; and
determining the prescription through a logic puzzle.

12. A system for measuring a pupillary distance, comprising:
a processor communicatively coupled to a server;
an application-program interface configured to receive an electronic image;
a physical memory communicatively coupled to the processor, wherein the physical memory includes a software, and wherein the software and the processor are configured to measure, from the electronic image, the pupillary distance based on an iris width.

13. The system of claim 12, wherein the processor is further configured to determine the pupillary distance based on a bowtie measurement.

14. The system of claim 13, wherein the bowtie measurement includes ten or more iterations.

15. The system of claim 14, wherein the bowtie measurement determines pixel widths of an iris based on changes in brightness.

16. The system of claim 14, wherein the processor is further configured to smooth the iris width and to remove outlier data.

17. The system of claim 12, further comprising a database of prescription data, wherein the application-program interface is configured to receive a prescription and analyze the prescription through logic puzzles to build a full prescription.

18. The system of claim 12, further comprising a database of prescription data, wherein the system is configured to receive a prescription and return a recommendation of an available eyewear frames based on the pupillary distance and the prescription.

19. The system of claim 18, wherein the pupillary distance is determined based on the formula:
$$d = a R - b {R}^{2}$$
where d is the iris width, a and b are coefficients, and R is the ratio of the pupillary distance to the iris width.

20. The system of claim 18, wherein the electronic image is a selfie photo.
